(19) United States
(12) Patent Application Publication
(10) Pub. No.: US 2003/0219448 A1
(43) Pub. Date: Nov. 27, 2003

(54) Peptide epitope-based vaccine for treating herpes simplex virus infections and related diseases

(75) Inventors: Lbachir BenMohamed, Arcadia, CA (US); Anthony B. Nesburn, Malibu, CA (US)

Correspondence Address: Richard H. Zaitlen, PILLSBURY WINTHROP LLP, Suite 2800, 725 South Figueroa Street, Los Angeles, CA, 90017-5406, US

(73) Assignee: CEDARS-SINAI MEDICAL CENTER

(21) Appl. No.: 10/213,053

(22) Filed: Aug. 6, 2002

Related U.S. Application Data
(60) Provisional application No. 60/383,170, filed on May 24, 2002.

(51) Int. Cl. A01N 37/18; A61K 38/00; A61K 39/12; A61K 39/245; A61K 39/255; A61K 39/265; A61K 39/27

(52) U.S. Cl. 424/186.1; 424/229.1; 514/2

(57) ABSTRACT

Described herein are peptide epitopes effective in the treatment of herpes simplex virus (HSV), as well as vaccines and other therapeutic compositions including the same. In various embodiments, the compositions of the present invention may include a pharmaceutically acceptable adjuvant to enhance the delivery and/or pharmacological efficacy of the epitope. Also described are methods for treating and preventing HSV with the aforementioned epitopes, such as by administering a vaccine including the same. Other methods describe the use of the TEPITOPE algorithm to identify epitopes that may be useful in the treatment of HSV and related or unrelated disease conditions.

### RELATED APPLICATIONS

This application claims the benefit of priority under 35 U.S.C. § 119(e) of U.S. provisional application serial No. 60/383,170, filed May 24, 2002, the contents of which are hereby incorporated by reference in their entirety.

### FIELD OF THE INVENTION

Embodiments of the present invention are directed to a composition and methods for treating and preventing Herpes Simplex Virus infection, based upon peptide epitopes.

### BACKGROUND OF THE INVENTION

The incidence of Herpes Simplex Virus (HSV) has risen 30 percent since the 1970's. One in four adults has HSV, and there are an estimated one million new cases of this disease every year. HSV infections have been associated with a spectrum of clinical syndromes including cold sores, genital lesions, corneal blindness and encephalitis. The percentage of infected persons who are not cognizant of their own infection with HSV is over 50% largely because these individuals either do not express the classic symptoms (e.g., they remain asymptomatic) or because they dismiss HSV as merely an annoying itch or rash in those cases in which the disease has external manifestations. Additionally, HSV may be treated, but clinical research has yet to identify a cure. Therefore, one cannot rid himself of HSV once infected; one can merely attempt to control infection when it reactivates. However, despite the increase of HSV prevalence during the last three decades, an effective vaccine that could help to control this epidemic is still not available.

There are two forms of herpes, commonly known as HSV-1 and HSV-2. Although HSV-1 is frequently associated with cold sores and HSV-2 with genital herpes, the viruses have many similarities and can infect either area of the body. HSV-specific B-cell and T-cell responses have been detected in humans during natural infection, yet latent infection and reactivation of HSV from peripheral ganglia and re-infection of the mucocutaneous tissues occurs frequently, causing recurrent ocular, labial or genital lesions. Other symptoms may include herpes keratitis, fever blisters, eczema herpeticum, cervical cancer, throat infections, rash, meningitis, nerve damage, and widespread infection in debilitated patients.

A variety of traditional vaccine strategies have been explored to induce protective immunity against HSV and recurrences. Live, attenuated, and killed viruses have been shown to provide protective immunity in murine HSV model systems, and recent HSV vaccine development has focused on various forms of recombinant expressed virus coat glycoprotein. Immunization with Freund's adjuvant-emulsified viral coat glycoproteins of either HSV-1 or HSV-2 provides complete or partial protective immunity against infection with both types of HSV in murine models. However, vaccine trials in human subjects with alum-absorbed glycoprotein D (gD) protein or with both glycoprotein B (gB) and gD proteins emulsified with MF59 adjuvant have had only marginal success in reducing recurrent genital shedding and disease. The antibody response to these vaccines has been shown as similar to natural HSV infections, yet these vaccines have been thus far unable to induce a Th1-like CD4⁺ T-cell response; this response is believed to be responsible for protection against HSV, at least in animal models.

Among other challenges that have prevented the development of an effective HSV vaccine are heretofore unidentified immunogenic epitopes (i.e., the portion of an Ag that binds to an antibody paratope, or that is presented on the surface of antigen presenting cells to T-cells, thereby triggering an immune response), the uncertainty about the exact immune correlates of protection, and the development of an efficient and safe immunization strategy. There is accumulating evidence in both animal and human models that CD4⁺ T-cell immunity is somehow related to the control of HSV infection, despite the fact that research has focused on antibody (Ab) and CD8⁺ T-cell responses. Therefore, activation of HSV-specific CD4⁺ Th-cells through the glycoproteins to which they react may be the basis for an effective vaccination protocol.

T-cells tend to recognize only a limited number of discrete epitopes on a protein Ag. In theory, numerous potential T-cell epitopes could be generated from a protein Ag. However, traditional approaches for identifying such epitopes from among the often hundreds or thousands of amino acids that cover the entire sequence of a protein Ag have used overlapping synthetic peptides (overlapping peptide method), which is inconvenient at best. In addition, progress on the mapping of T-cell epitopes has been slow due to reliance on studies of clones, an approach that generally involves extensive screening of T-cell precursors isolated from whole Ag-stimulated cells.

Another alternative to cloning T-cells employs tetramer-guided epitope mapping, which provides a straightforward cloning of the Ag-specific T-cells through single-cell sorting. However, in addition to requiring formation of pools of overlapping peptides, there are concerns that relevant peptides present in these pools will be competed out by irrelevant peptides. Furthermore, the relative instability of Major Histocompatibility Complex (MHC) class II tetramers (when compared to MHC class I tetramers) underscores that the tetramer approach still needs improvement.

Other, relatively laborious strategies have been used to identify small subsets of candidate epitopes by sequencing peptides eluted from purified MHC molecules from pathogen infected cells and then testing their MHC binding affinity. High affinity peptides are then tested for their ability to induce pathogen-specific T-cells. The major drawback of these approaches is the number of peptide sequences that need to be synthesized and tested, thus rendering them expensive, labor-intensive and time-consuming.

In an attempt to overcome these obstacles, several studies have implemented the recently-developed TEPITOPE prediction algorithm (Available from Dr. Juergen Hammer, Roche Discovery Technologies, Hoffmann-La Roche, Nutley, N.J.) to identify potential T-cell epitopes within protein Ags. This algorithm can be used to predict and create immunogenic peptide sequences and has been implemented with such protein Ags as bacterial Ags, tumor Ags and allergens. In addition, the peptide-based approach offers several potential advantages over the conventional practice of using whole proteins, in terms of purity, lot-to-lot consistency, cost of production and a high specificity in eliciting immune responses.

Yet even if T-cell epitopes could be accurately predicted and synthesized using the TEPITOPE algorithm, peptide-based vaccines still face limitations of weak immunogenicity, coupled with a paucity of sufficiently potent adjuvants that can be tolerated by humans. Large numbers of adjuvants are known to enhance both B-cell and T-cell responses in laboratory animals, but adjuvants compatible to humans are limited due to their toxic effects. The aluminum hydroxide salts (ALUM) are the only adjuvants widely used in human vaccines, but ALUM-adsorbed Ags preferentially induce Th2 responses as opposed to Th1 responses believed to be needed to increase the efficiency of a CD4⁺ T-cell immune response; especially advantageous in an HSV treatment.

There is therefore a need in the art for peptide epitopes and vaccines incorporating the same that are safe and effective in humans and other mammals in treating and/or providing protective immunity against HSV infection. Such peptide epitopes could have a dramatic impact on the health of humans and other mammals worldwide; a vaccine being a particularly advantageous pharmacological formulation for the therapeutic delivery thereof.

### SUMMARY OF THE INVENTION

Disclosed herein are peptide epitopes useful in the treatment or prevention of HSV. These epitopes may be administered to a mammal by any conventional means, such as, by way of example, a vaccine composition. Compositions incorporating the epitopes of the present invention may further include a pharmaceutical carrier and/or an adjuvant, to provide a therapeutically convenient formulation and/or to enhance biochemical delivery and efficacy of the epitopes. Methods of treating or preventing HSV with the epitopes of the present invention are also provided.

Also disclosed herein is a method of identifying HSV-1 gD-derived peptides bearing potent CD4⁺ T-cell epitopes and evaluating the peptides' vaccine potential using a clinically suitable adjuvant. The HSV-1 gD-derived peptides identified in the context of HSV infection, together with the peptides' observed function, may be the basis of an immuno-prophylactic or immuno-therapeutic vaccine for HSV primary infection and recurrences.

### BRIEF DESCRIPTION OF THE DRAWINGS

FIG. 1 is a graphical representation of the proliferative responses generated by gD peptides predicted from the TEPITOPE algorithm in accordance with an embodiment of the present invention. Peptide concentration was measured in μM.

FIG. 2 depicts a fluorescent activated cell sorter (FACS) analysis of stimulated cells graphically depicted in FIG. 1 in accordance with an embodiment of the present invention. Most responding cells were of CD4⁺ phenotype.

FIG. 3 is a graphical representation of the proliferative responses generated by each of the dominant gD peptides predicted from the TEPITOPE algorithm in accordance with an embodiment of the present invention. Peptide concentration was measured in μM.

FIG. 4 is a graphical representation of cytokine secretion elicited by gD peptides predicted from the TEPITOPE algorithm in accordance with an embodiment of the present invention.

FIG. 5 is a graphical representation of ³H Thymidine uptake in accordance with an embodiment of the present invention. FIG. 5A depicts ³H Thymidine uptake by ultraviolet-inactivated HSV-1, and FIG. 5B depicts ³H Thymidine uptake by ultraviolet-inactivated HSV-1 comparing HSV infected dendritic cells and HSV mock infected dendritic cells.

FIG. 6 is a graphical representation of ³H Thymidine uptake by gD peptides comparing HSV infected dendritic cells and HSV mock infected dendritic cells in accordance with an embodiment of the present invention.

### DETAILED DESCRIPTION OF THE INVENTION

The present invention is based on the surprising discovery of immunogenic glycoprotein D (gD) protein epitopes that can elicit potent CD4⁺ T-cell responses in animal models. While not wishing to be bound by any theory, it is believed that these epitopes induce the Th-1 subset of T-cells by the selective expansion of CD4⁺ T-cells and stimulation of IL-2 and IFN-γ; important cytokines in the elimination of HSV and the treatment of various other conditions. It is further believed that inducing the Th-1 subset of T-cells may substantially increase the modulation and maintenance of a memory immune response to HSV. Therefore, a therapeutic basis for an effective treatment and vaccination against HSV may be the activation of HSV-specific CD4⁺ Th-cells with the protein epitopes of the present invention.

As used herein, “treatment” includes, but is not limited to, ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

The following twelve gD peptide epitopes have been identified and are implemented in accordance with various embodiments of the present invention: gD₁₋₂₉, gD₂₂₋₅₂, gD₄₉₋₈₂, gD₇₇₋₁₀₄, gD₉₆₋₁₂₃, gD₁₂₁₋₁₅₂, gD₁₄₆₋₁₇₉, gD₁₇₆₋₂₀₆, gD₂₀₀₋₂₃₄, gD₂₂₈₋₂₅₇, gD₂₈₇₋₃₁₇, and gD₃₃₂₋₃₅₈. Protein sequences corresponding to these epitopes are included herein as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and SEQ ID NO: 12, respectively. These peptide epitopes, either alone or in combination with one another, may be useful in the treatment of HSV-1 and/or HSV-2 primary infections and recurrences and related disease conditions including, but in no way limited to, cold sores, genital lesions, corneal blindness, and encephalitis, and any other disease or pathological condition in which expansion of CD4⁺ T-cells, stimulation of IL-2 or IFN-γ, and/or the induction of the Th-1 subset of T-cells may be desirable (all of which are hereinafter included in the term “epitope-sensitive condition”).

Ten of the epitopes of the present invention belong to the external N-terminal portion of gD (SEQ ID NOS: 1-10); one lies adjacent to the hydrophobic membrane anchorage domain of gD (SEQ ID NO: 11); and one is part of the proposed hydrophilic C-terminal cytoplasmic portion of gD (SEQ ID NO: 12). Of these epitopes, six mapped to non-glycosylated regions of gD (SEQ ID NO: 1, SEQ ID NO: 3, SEQ ID NO: 7, SEQ ID NO: 10, SEQ ID NO: 11 and SEQ ID NO: 12).

In a first aspect of the present invention, a vaccine strategy against an epitope-sensitive condition includes the administration to a mammal of any of the peptide epitopes described herein either alone or in any suitable combination either with one another or with additional peptide epitopes not specifically enumerated herein as would be readily recognized by one of skill in the art. gD protein is conventionally administered to ameliorate the symptoms of HSV, and to thereby slow or halt the spread of HSV disease; although the gD peptides of the present invention may additionally be used in the prevention of HSV infection (e.g., as a prophylactic vaccine). Thus, in various embodiments of the present invention, the epitopes may be administered in a multi-component immuno-therapeutic (i.e., to treat the disease) and/or an immuno-prophylactic (i.e., to prevent the disease) vaccine, effective against HSV and/or other epitope-sensitive conditions. In particular, the gD peptides of the present invention may provide at least partial, and in some cases full protective immunity to HSV and other epitope-sensitive conditions, and may thereby function as a preventative vaccination.

Moreover, in another aspect of the present invention, any of the peptides represented by SEQ ID NOS: 1-12, any peptide including one or more of the peptides represented by SEQ ID NOS: 1-12, any portion of the peptides represented by SEQ ID NOS: 1-12 or combinations thereof may be incorporated into a vaccine effective in the treatment of HSV, or into another epitope-based vaccine. In alternate embodiments of the present invention, one may implement one or more of the peptide epitopes of the present invention, but, to obtain a desired clinical result, one may not need to utilize the entire sequence. In fact, a portion of one or more of the peptides represented by SEQ ID NOS: 1-12 may be clinically effective. In still further embodiments of the present invention, one may include one or more of the peptide epitopes of the present invention represented by SEQ ID NOS: 1-12 in a larger protein molecule. Doing so may be advantageous for any number of reasons, as will be readily recognized by one of skill in the art. Including one of the peptide epitopes in such a larger molecule is also contemplated as being within the scope of the present invention.

As used herein, the term “vaccine” refers to any combination of peptides or single peptide formulation. There are various reasons why one might wish to administer a vaccine of a combination of the peptides of the present invention rather than a single peptide. Depending on the particular peptide that one uses, a vaccine might have superior characteristics as far as clinical efficacy, solubility, absorption, stability, toxicity and patient acceptability are concerned. It should be readily apparent to one of ordinary skill in the art how one can formulate a vaccine of any of a number of combinations of peptides of the present invention. There are many strategies for doing so, any one of which may be implemented by routine experimentation. For example, one can survey specific patient MHC restriction or test different combinations, as illustrated in the ensuing Example 13.

The peptides of the present invention may be administered as a single agent therapy or in addition to an established therapy, such as inoculation with live, attenuated, or killed virus, or any other therapy known in the art to treat HSV or another epitope-sensitive condition.

The appropriate dosage of the peptides of the invention may depend on a variety of factors. Such factors may include, but are in no way limited to, a patient's physical characteristics (e.g., age, weight, sex), whether the compound is being used as single agent or adjuvant therapy, the type of MHC restriction of the patient, the progression (i.e., pathological state) of the HSV infection or other epitope-sensitive condition, and other factors that may be recognized by one skilled in the art. In general, an epitope or combination of epitopes may be administered to a patient in an amount of from about 50 micrograms to about 5 mg; dosage in an amount of from about 50 micrograms to about 500 micrograms is especially preferred.

One may administer a vaccine of the present invention by any suitable method, which may include, but is not limited to, systemic injections (e.g., subcutaneous injection, intradermal injection, intramuscular injection, intravenous infusion) mucosal administrations (e.g., nasal, ocular, oral, vaginal and anal formulations), topical administration (e.g., patch delivery), or by any other pharmacologically appropriate technique. Vaccination protocols using a spray, drop, aerosol, gel or sweet formulation are particularly attractive and may be also used. The vaccine may be administered for delivery at a particular time interval, or may be suitable for a single administration. In those embodiments wherein the composition of the present invention is formulated for administration at a delivery interval, it is preferably administered once every 4 to 6 weeks.

Vaccines of the invention may be prepared by combining at least one peptide with a pharmaceutically acceptable liquid carrier, a finely divided solid carrier, or both. As used herein, “pharmaceutically acceptable carrier” refers to a carrier that is compatible with the other ingredients of the formulation and is not toxic to the subjects to whom it is administered. Suitable such carriers may include, for example, water, alcohols, natural or hardened oils and waxes, calcium and sodium carbonates, calcium phosphate, kaolin, talc, lactose, combinations thereof and any other suitable carrier as will be recognized by one of skill in the art. In a most preferred embodiment, the carrier is present in an amount of from about 10 μL (micro-Liter) to about 100 μL.

In a preferred embodiment, the vaccine composition includes an adjuvant; most preferably, Montanide ISA720 (M-ISA-720; available from Seppic, Fairfield, N.J.), an adjuvant based on a natural metabolizable oil. As further described in the ensuing examples, M-ISA-720 was found to enhance a significant HSV-specific Th1 CD4⁺ T-cell response, and the subcutaneous injection of vaccine formulated with the same was well-tolerated by recipients. Compositions of the present invention preferably include from about 15 μL to about 25 μL M-ISA-720.

In various embodiments, vaccines according to the invention may be combined with one or more additional components that are typical of pharmaceutical formulations such as vaccines, and can be identified and incorporated into the compositions of the present invention by routine experimentation. Such additional components may include, but are in no way limited to, excipients such as the following: preservatives, such as ethyl-p-hydroxybenzoate; suspending agents such as methyl cellulose, tragacanth, and sodium alginate; wetting agents such as lecithin, polyoxyethylene stearate, and polyoxyethylene sorbitan mono-oleate; granulating and disintegrating agents such as starch and alginic acid; binding agents such as starch, gelatin, and acacia; lubricating agents such as magnesium stearate, stearic acid, and talc; flavoring and coloring agents; and any other excipient conventionally added to pharmaceutical formulations.

Further, in various embodiments, vaccines according to the invention may be combined with one or more of the group consisting of a vehicle, an additive, a pharmaceutical adjunct, a therapeutic compound or agent useful in the treatment of HSV, and combinations thereof.

In another aspect of the present invention, a method of creating a vaccine is provided. The method may include identifying an immunogenic epitope; synthesizing a peptide epitope from the immunogenic epitope; and creating a composition that includes the peptide epitope in a pharmaceutical carrier. The composition may have characteristics similar to the compositions described above in accordance with alternate embodiments of the present invention.

As further described in the ensuing Examples, the TEPITOPE algorithm (Available from Dr. Juergen Hammer, Roche Discovery Technologies, Hoffmann-La Roche, Nutley, N.J.) may be implemented in accordance with the compositions and methods of the present invention to identify the epitopic regions of the HSV-1 gD; although various other epitope prediction software programs commercially or otherwise available may be used to predict immunogenic epitopes, as will be readily recognized by those of skill in the art. Using the TEPITOPE algorithm, the twelve regions of the HSV-1 gD bearing putative antigenic and immunogenic determinants were detected within a stringent threshold (SEQ ID NOS: 1-12), and as depicted in Table 1.

TABLE 1

Peptide bearing potential T-cell epitopes identified within the HSV-1
glycoprotein D (g^{D}) using the TEPITOPE algorithm

| | | | | |
|---|---|---|---|---|
| | | | | Nber^{(c)} |
| SEQ ID. | Peptide | Sequence^{(a)} | MW^{(b)} | aa |

| | | | | |
|---|---|---|---|---|
| 1. | g^{D}₁₋₂₉ | SKYALVDASLKMADPNRFRGKDLPVLDQL | 2260 | 29 |
| 2. | g^{D}₂₂₋₅₂ | DLPVLQLTDPPGVRRVYHIQAGLPDPFQPPS | 3422 | 31 |
| 3. | g^{D}₄₉₋₈₂ | QPPSLPITVYYAVLERACRSVLLNAPS EAPQIVR | 3750 | 34 |
| 4. | g^{D}₇₇₋₁₀₄ | APQIVRGASEDVRKQPYNLTIAWFRMGG | 3160 | 28 |
| 5. | g^{D}₉₆₋₁₂₃ | TIAWFRMGGNCAIPITVMEYTECSYNKS | 3183 | 28 |
| 6. | g^{D}₁₂₁₋₁₅₂ | NKSLGACPIRTQPRWNYYDSFSAVSEDNLGFL | 3648 | 32 |
| 7. | g^{D}₁₄₆₋₁₇₉ | EDNLGFLMHAPAFETAGTYLRLVKINDWTEITQF | 3941 | 34 |
| 8. | g^{D}₁₇₆₋₂₀₆ | ITQFILEHRAKGSCKYALPLRIPPSACLSPQ | 3436 | 31 |
| 9. | g^{D}₂₀₀₋₂₃₄ | SACLSPQAYQQGVTVDSIGMLPRFIPENQRTVAVY | 3838 | 35 |
| 10. | g^{D}₂₂₈₋₂₅₇ | QRTVAVYSLKIAGWHGPKAPYTSTLLPPEL | 3293 | 30 |
| 11. | g^{D}₂₈₇₋₃₁₇ | APQIPPNWHIPSIQDAATPYHPPATPNNMGL | 3345 | 31 |
| 12. | g^{D}₃₃₂₋₃₅₈ | ICGIVYWMRRHTQKAPKRIRLPHIRED | 3372 | 27 |

^{(a)}The amino acid sequence of identified peptides represented in single-letter code. The peptides were synthesized based on the HSV-1 glycoprotein D sequence (strain 17)
^{(b)}Molecular weight (MW).
^{(c)}Number of amino acids.

While not wishing to be bound by any theory, it is believed that the regions obtained from the analysis are likely to be less constrained than other parts of the molecule, thus rendering them more accessible to proteolysis; an event that precedes T-cell epitope presentation in association with MHC molecules.

### EXAMPLES

The following examples are typical of the procedures that may be used to treat patients suffering from HSV, or to evaluate the efficacy of the vaccination strategy which may be used to treat such patients in accordance with various embodiments of the present invention. Modifications of these examples will be readily apparent to those skilled in the art who seek to treat patients whose condition differs from those described herein.

### Example 1

T-cell Epitope Prediction Using TEPITOPE

The glycoprotein D (gD) sequence (strain 17) was loaded into prediction software (TEPITOPE) to predict promiscuous epitopes. The TEPITOPE algorithm is a WINDOWS (Microsoft Corporation, Redmond, Wash.) application that is based on 25 quantitative matrix-based motifs that cover a significant part of human, human leukocyte antigen (HLA) class II peptide binding specificity. Starting from any protein sequence, the algorithm permits the prediction and parallel display of ligands for each of the 25 HLA-DR alleles. The TEPITOPE prediction threshold, which was set at 5%, predicted twelve regions (SEQ ID NOS: 1-12) that would bind at least 50% of the MHC class II molecules.

### Example 2

Synthesis of Peptides

A total of 12 gD peptides, each consisting of 27 to 34 amino acids, were synthesized by BioSource International (Hopkinton, Mass.) on a 9050 Pep Synthesizer Instrument using solid phase peptide synthesis (SPPS) and standard F-moc technology (PE Applied Biosystems, Foster City, Calif.). Peptides were cleaved from the resin using Trifluoroacetic acid:Anisole:Thioanisole:Anisole:EDT:Water (87.5:2.5:2.5:2.5:5%) followed by ether extraction (methyl-t-butyl ether) and lyophilization. The purity of peptides was greater than 90%, as determined by reversed phase high performance liquid chromatography (RP-HPLC) (VYDAC C18) and mass spectrometry (VOYAGER MALDI-TOF System). Stock solutions were made at 1 mg/ml in water, except for peptide gD₁₄₆₋₁₇₉ that was solubilized in phosphate buffered saline (PBS). All peptides were aliquoted, and stored at −20° C. until assayed. Studies were conducted with the immunogen emulsified in M-ISA-720 adjuvant (Seppic, Fairfield, N.J.) at a 3:7 ratio and immediately injected into mice.

### Example 3

Preparation of Herpes Simplex Virus Type 1

The McKrae strain of HSV-1 was used in this study. The virus was triple plaque purified using classical virology techniques. UV-inactivated HSV-1 (UV-HSV-1) was made by exposing the live virus to a Phillips 30 W UV bulb for 10 min at a distance of 5 cm. HSV inactivation in this manner was ascertained by the inability of UV-HSV-1 to produce plaques when tested on vero cells.

### Example 4

Immunization in Animal Models

Six to eight week old C57BL/6 (H-2^{b}), BALB/c (H-2^{d}), and C3H/HeJ (H-2^{k}) mice (The Jackson Laboratory, Bar Harbor, Me.) were used in all experiments. Groups of five mice per strain, were immunized subcutaneously with peptides in M-ISA 720 adjuvant on days 0 and 21. In an initial experiment the optimal dose response to peptide gD₁₋₂₉ was investigated and no significant differences were found among doses of 50, 100 and 200 μg. Subsequent experiments used 100 μg (at day 0) and 50 μg (at day 21) of each peptide in a total volume of 100 μl. Under identical conditions control mice received the adjuvant alone, for control purposes.

### Example 5

Pep tide-specific T-cell Assay

Twelve days after the second immunization, spleen and inguinal lymph nodes (LN) were removed and placed into ice-cold serum free HL-1 medium supplemented with 15 mM HEPES, 5×10⁻⁵ M β-mercaptoethanol, 2 mM glutamine, 50 U of penicillin and 50 μg of streptomycin (GIBCO-BRL, Grand Island, N.Y.) (complete medium, CM). The cells were cultured in 96-well plates at 5×10⁵ cells/well in CM, with recall or control peptide at 30,10, 3, 1, or 0.3 μg/ml concentration, as previously described in (BenMohamed et al., 2000 and 2002). The cell suspensions were incubated for 72 h at 37° C. in 5% CO₂. One μCi (micro-curie) of (³H)-thymidine (Dupont NEN, Boston, Mass.) was added to each well during the last 16 h of culture. The incorporated radioactivity was determined by harvesting cells onto glass fiber filters and counted on a Matrix 96 direct ionization-counter (Packard Instruments, Meriden, Conn.). Results were expressed as the mean cpm of cell-associated (³H)-thymidine recovered from wells containing Ag minus the mean cpm of cell-associated (³H)-thymidine recovered from wells without Ag (Δ cpm) (average of triplicate). The Stimulation Index (SI) was calculated as the mean cpm of cell-associated (³H)-thymidine recovered from wells containing Ag divided by the mean cpm of cell-associated (³H)-thymidine recovered from wells without Ag (average of triplicate). For all experiments the irrelevant control peptide gB₁₄₁₋₁₆₅ and the T-cell mitogen Concanavalin A (ConA) (Sigma, St. Louis, Mo.) were used as negative and positive controls, respectively. Proliferation results were confirmed by repeating each experiment twice. A T-cell proliferative response was considered positive when Δ cpm>1000 and SI>2.

### Example 6

Cytokine Analysis

T-cells were stimulated with either immunizing peptides (10 μg/ml), the irrelevant control peptide (10 μg/ml), UV-inactivated HSV-1 (MOI=3), or with ConA (0.5 μg/ml) as a positive control. Culture media were harvested 48 h (for IL-2) or 96 h (for IL-4 and IFN-γ) later and analyzed by specific sandwich ELISA following the manufacturer's instructions (PharMingen, San Diego, Calif.).

### Example 7

Flow Cytometric Analysis

The gD peptide stimulated T-cells were phenotyped by double staining with anti-CD4⁺ and anti-CD8⁺ monoclonal antibodies (mAbs) and analyzed by FACS. After 4 days stimulation with 10 μM of each peptide, one million cells were washed in cold PBS-5% buffer and incubated with phycoerythrin (PE) anti-CD4 (Pharmingen, San Diego, Calif.) or with FITC anti-CD8⁺ (Pharmingen, San Diego, Calif.) mAbs for 20-30 min on ice. Propidium iodide was used to exclude dead cells. For each sample, 20,000 events were acquired on a FACSCALIBUR and analyzed with CELLQUEST software (Becton Dickinson, San Jose, Calif.), on an integrated POWER MAC G4 (Apple Computer, Inc., Cupertino, Calif.).

### Example 8

Derivation of Bone Marrow Dendritic Cells

Murine bone marrow-derived dendritic cells (DC) were generated using a modified version of the protocol as described previously in (BenMohamed et al., 2002). Briefly, bone marrow cells were flushed out from tibias and femurs with RPMI-1640, and a single cell suspension was made. A total of 2×10⁶ cells cultured in 100-P tissue dishes containing 10 ml of RPMI-1640 supplemented with 2 mM glutamine, 1% non-essential amino acids (Gibco-BRL), 10% fetal calf serum, 50 ng/ml granulocyte macrophage colony stimulatory factor (GM-CSF) and 50 ng/ml IL-4 (PeproTech Inc, Rocky Hill, N.J.). Cells were fed with fresh media supplemented with 25 ng/ml GM-CSF and 25 ng/ml IL-4 every 72 hrs. After 7 days of incubation, this protocol yielded 50-60×10⁶ cells, with 70 to 90% of the non-adherent-cells acquiring the typical morphology of DC. This was routinely confirmed by FACS analysis of CD11c, class II and DEC-205 surface markers of DC.

### Example 9

CD4⁺ T-cell Responses to HSV Infected DC

Approximately 10⁵ purified CD4⁺ T-cells were derived by stimulation twice biweekly with 5×10⁵ irradiated DC pulsed with recall peptides. The CD4⁺ T-cell effector cells were incubated with X-ray-irradiated DC (T:DC=50:1) that were infected with UV-HSV-1 (3, 1, 0.3. 0.1 multiplicity of infection (MOI)). As control, CD4⁺ T-cells were also incubated with mock infected DC. The DC and CD4⁺ T-cells were incubated for 5 days at 37° C. and (³H)-thymidine was added to the cultures 18 hrs. before harvesting. Proliferative responses were tested in quadruplicated wells, and the results were expressed as mean cpm±SD. In some experiments splenocytes from immunized or control mice were re-stimulated in vitro by incubation with heat-inactivated or UV-inactivated HSV-1.

### Example 10

Infection and In Vivo Depletion of CD4⁺ and CD8⁺ T-cells

Mice were infected with 2×10⁵ pfu per eye of HSV-1 in tissue culture media administered as an eye drop in a volume of 10 μl. Beginning 21 days after the second dose of peptide vaccine, some mice were intraperitoneally injected with six doses of 0.1 ml of clarified ascetic fluid in 0.5 ml of PBS containing mAb GK1.5 (anti-CD4) or mAb 2.43 (anti-CD8) on day −7, −1, 0, 2, and 5 post-infection. Flow cytometric analysis of spleen cells consistently revealed a decrease in CD4⁺ and CD8⁺ T-cells in such treated mice to levels of <3% compared to that of normal mice.

### Example 11

Statistical Analysis

Figures represent data from at least two independent experiments. The data are expressed as the mean±SEM and compared by using Student's t test on a STATVIEW II statistical program (Abacus Concepts, Berkeley, Calif.).

### Example 12

Prediction of gD Epitopes that Elicit Potent CD4⁺ T-cell Responses in Mice with Diverse MHC Backgrounds

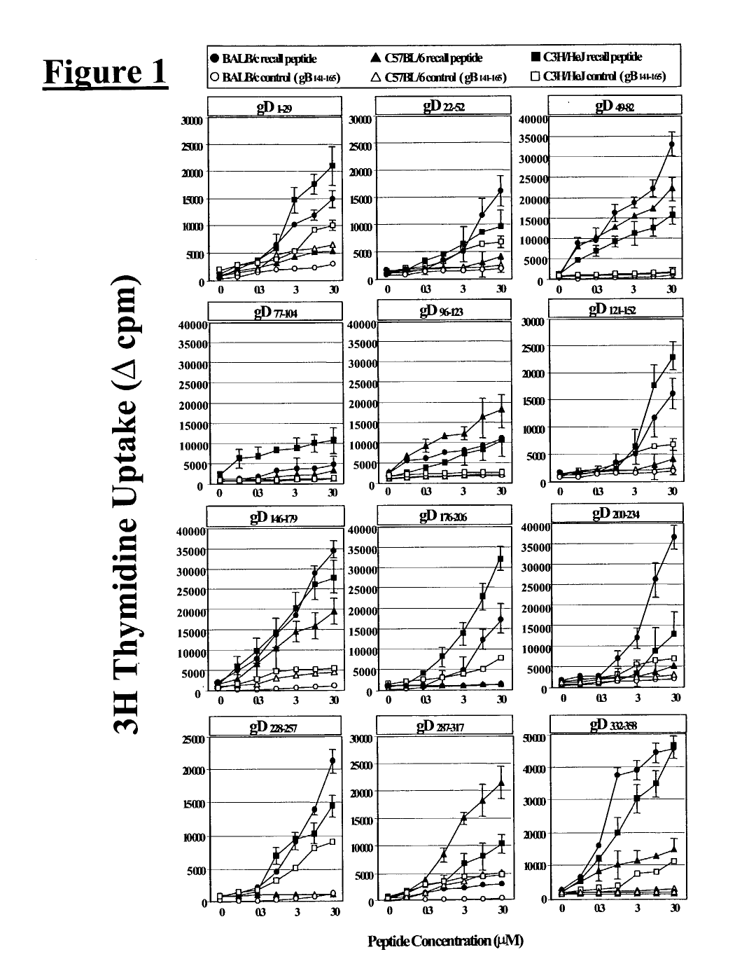

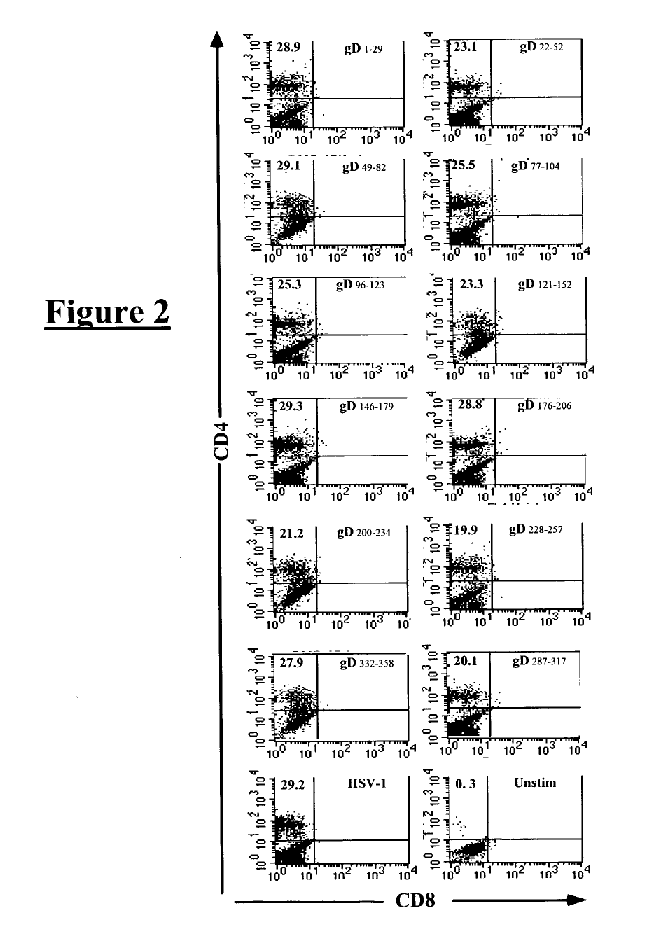

The selected peptides were used to immunize H2^{b}, H-2^{d} and H-2^{k} mice and peptide-specific T-cell proliferative responses were determined from spleen and lymph node (LN) cells. Depending on the peptides and strain of mice used, significant proliferative responses were generated by every gD peptide. Thus, each of the twelve chosen regions contained at least one T-cell epitope (FIG. 1). The strongest T-cell responses were directed primarily, although not exclusively, to five peptides (gD₁₋₂₉, gD₄₉₋₈₂, gD₁₄₆₋₁₇₉, gD₂₂₈₋₂₅₇, and gD₃₃₂₋₃₅₈). The dominant T-cell responses of H-2^{b}, H2^{d} and H-2^{k} mice were focused on the same three peptides (gD₄₉₋₈₂, gD₁₄₆₋₁₇₉ and gD₃₃₂₋₃₅₈), suggesting that they contain major T-cell epitopes (FIG. 1). In contrast, gD₂₀₀₋₂₃₄ and gD₂₂₈₋₂₅₇ appeared to be genetically restricted to H2^{d} mice. The levels of response were relatively high with a Δ cpm≧10,000 for most peptides and up to 50,000 cpm for gD₃₃₂₋₃₅₈ (FIG. 1). Although relatively moderate compared to the remaining gD peptides, the responses to gD₂₂₋₅₂, gD₇₇₋₁₀₄, and gD₉₆₋₁₂₃ were also significant (FIG. 1).

The specificity of the proliferative responses was ascertained by the lack of responses after re-stimulation of immune cells with an irrelevant peptide (gB₁₄₁₋₁₆₅) (FIG. 1), and the lack of response to any of the gD peptides in adjuvant-injected control mice (data not shown). FACS analysis of stimulated cells indicated that most responding cells were of CD4⁺ phenotype (FIG. 2). As expected, these responses were blocked by a mAb against CD4⁺ molecules as depicted in Table 2, but not by a mAb against CD8⁺.

TABLE II
CD4⁺ dependence of T-cell proliferation and cytokine secretion induced by gD peptides^{(a)}

| | T-cell proliferation (SI)^{(b)(c)} | | | IL-2 (pg/ml)^{(c)} | | | IFNγ(ng/ml)^{(c)} | | |
|---|---|---|---|---|---|---|---|---|---|
| Antigen | None | anti-CD4 | anti-CD8 | None | anti-CD4 | anti-CD8 | None | anti-CD4 | anti-CD8 |
| gD₁₋₂₉ | 8(+/−1) | 1(+/−1) | 7(+/−2) | 45(+/−3) | 12(+/−2) | 47(+/−1) | ‘13(+/−1) | 5(+/−3) | 11(+/−2) |
| gD₄₉₋₈₉ | 13(+/−2) | 2(+/−1) | 16(+/−2) | 92(+/−5) | 22(+/−2) | 88(+/−5) | 60(+/−4) | 6(+/−2) | 66(+/−2) |
| gD₃₃₂₋₃₅₈ | 16(+/−2) | 3(+/−2) | 16(+/−1) | 135(+/−6) | 36(+/−1) | 130(+/−4) | 179(+/−1) | 4(+/−1) | 54(+/−1) |
| UV-HSV | 6(+/−1) | 3(+/−2) | 7(+/−) | 87(+/−6) | 16(+/−1) | 76(+/−4) | 133(+/−3) | 4(+/−1) | 66(+/−1) |

^{(a)}Splenocyte-derived T-cells were treated with no Abs (None). or with Abs to CD4 (anti-CD4⁺) or CD8 (anti-CD8⁺) molecules and stimulated with the indicated peptides or UV mactivated virus.
^{(b)}The Simulation Index (SI) was calculated as the mean cpm of cell-associated (³H)-thymidine recovered from wells containing Ag divided by the mean cpm of cell-associated (³H)-thymidine recovered from wells without Ag.
(c)Values represent average of data obtained from triplicates (+/− standard deviation).

Collectively, these results showed four new epitope sequences, gD₄₉₋₈₂, gD₁₄₆₋₁₇₉, gD₂₂₈₋₂₅₇ and gD₃₃₂₋₃₅₈, that contain major CD4⁺ T-cell sites of gD protein.

### Example 13

Simultaneous Induction of Multiple Ag-specific T-cells to Pools of gD-Derived Peptides

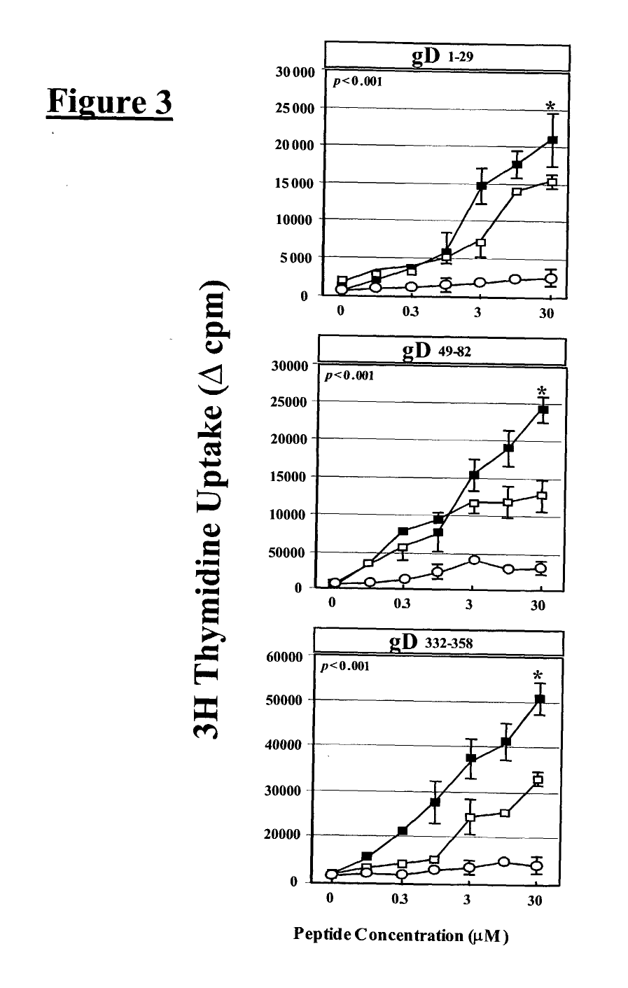

To fully exploit the potential advantages of the peptide-based vaccine approach, the ability of pools of gD peptides to simultaneously induce multiple T-cells specific to each peptide within the pool was explored (FIG. 3). In these experiments, the immunogenicity in H-2^{d} mice of mixed versus individual peptides was compared side by side to investigate if there was any agonistic or synergistic interaction between the peptide epitopes composing the pool. As a control, H-2^{d} mice were injected with M-ISA-720 alone. Immunization with pool of gD₁₋₂₉, gD₄₉₋₈₂, and gD₃₃₂₋₃₅₈ peptides generated multi-epitopic and significantly higher T-cell responses specific to each peptide (p<0.001) (FIG. 3). Thus, when evaluated individually, each peptide induced a relatively lower response (p<0.001) (FIG. 3). In a similar experiment, the responses induced by a pool of gD₉₆₋₁₂₃, gD₁₄₆₋₁₇₉, and gD₂₈₇₋₃₁₇ peptides were also at a higher level than the responses induced when individual peptides were employed (data not shown).

### Example 14

Determination of Subset of CD4⁺ T-cells Preferentially Induced by Peptides

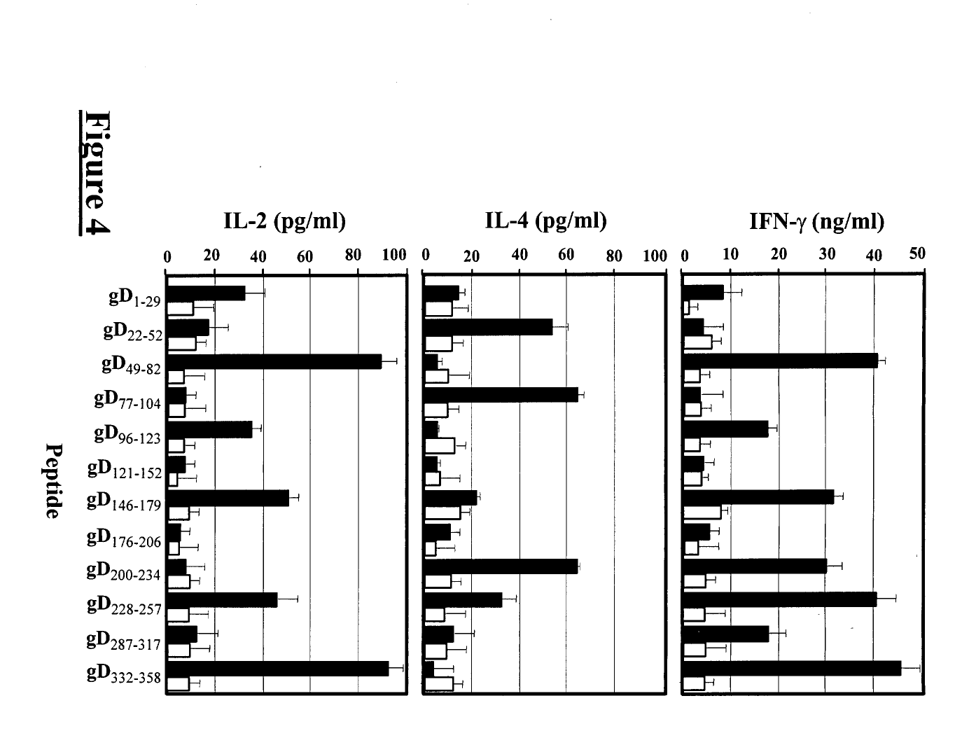

To determine the type of CD4⁺ T-helper cells involved in lymphocyte proliferation, the inventors studied the pattern of peptide-specific IL-2, IL-4 and IFN-γ cytokines induced by each gD peptide. As shown, the gD₁₋₂₉, gD₄₉₋₈₂, gD₉₆₋₁₂₃, gD₁₄₆₋₁₇₉, gD₂₂₈₋₂₅₇ and gD₃₃₂₋₃₅₈ peptides induced Th1 cytokines secretion more efficiently than the remaining peptides (FIG. 4). The gD₂₂₋₅₂ and gD₇₇₋₁₀₄ peptides preferentially induced Th-2 cytokines. The gD₂₀₀₋₂₃₄ peptide induced a mixed response since both IL-4 and IFN-γ were induced to a comparable extent (FIG. 4). Overall, for most peptides, the level of IL-2 and IFN-γ induced was consistently higher than the level of IL-4, indicating that the selected HSV-1 gD peptides emulsified in the M-ISA-720 adjuvant elicited a polarized Th-1 immune response (FIG. 4). Antibody blocking of T cell activity revealed that cytokines were mainly produced by CD4⁺ T-cells and only slightly by CD8⁺ T-cells (Table 2).

### Example 15

Determination of Whether T-cells Induced by gD-peptides are Relevant to the Native Viral Protein

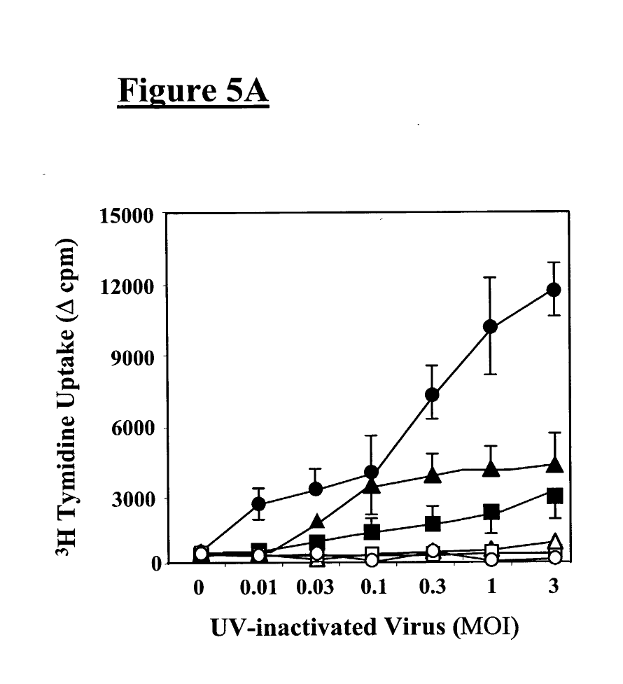

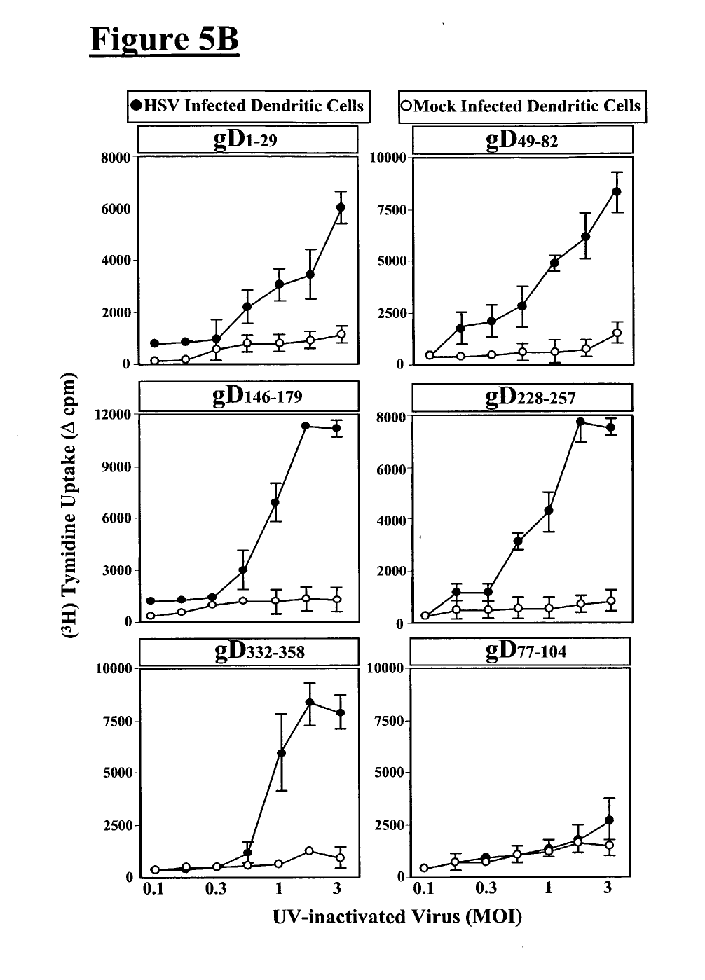

To ensure that the observed T-cell responses to the synthetic peptides were reactive to the naturally processed epitopes, the responses to HSV-1 were monitored. T-cells from H-2^{b}, H-2^{d} and H-2^{k} mice immunized with gD₄₉₋₈₂, gD₁₄₆₋₁₇₉, gD₂₂₈₋₂₅₇ and gD₃₃₂₋₃₅₈ showed significant proliferation (FIG. 5A) and IFN-γ secretion (Table 2) upon in vitro stimulation with UV-inactivated HSV-1. Under the same conditions, T-cells from the adjuvant-injected control mice did not respond to UV-HSV-stimulation (FIG. 5A). Thus, these responses were antigen specific and were not due to a mitogenic effect of viral particles. The HSV-1-specific T cell responses were strongly reduced by anti-CD4⁺ mAb treatment, but not by anti-CD8⁺ mAbs (Table 2).

Experiments were performed to determine if the CD4⁺ T-cells induced by gD peptides would recognize the naturally processed viral protein as presented by HSV-1 infected cells. The CD4⁺ T-cell lines specific to gD₁₋₂₉, gD₄₉₋₈₂, gD₁₄₆₋₁₇₉, gD₂₂₈₋₂₅₇ or gD₃₃₂₋₃₅₈, derived from H-2^{d} mice, responded upon in vitro stimulation with autologous UV-HSV infected bone marrow derived dendritic cells (DC) (FIG. 5B). No response was observed when mock infected autologous DC were employed as target cells (FIG. 5B). The CD4⁺ T-cells lines induced by gD₇₇₋₁₀₄ (FIG. 5B), as well as by gD₂₂₋₅₂, gD₁₂₁₋₁₅₂, gD₁₇₆₋₂₀₆ or gD₂₀₀₋₂₃₄ peptides (data not shown) failed to recognize UV-HSV-infected DC. Overall, these results indicated that processing and presentation of the epitopes contained in the gD₁₋₂₉, gD₄₉₋₈₂, gD₁₄₆₋₁₇₉, gD₂₂₈₋₂₅₇ and gD₃₃₂₋₃₅₈ peptides occurred in HSV infected cells.

### Example 16

Determination of Immunodominance in HSV-primed T-cell Responses to Selected gD-peptides

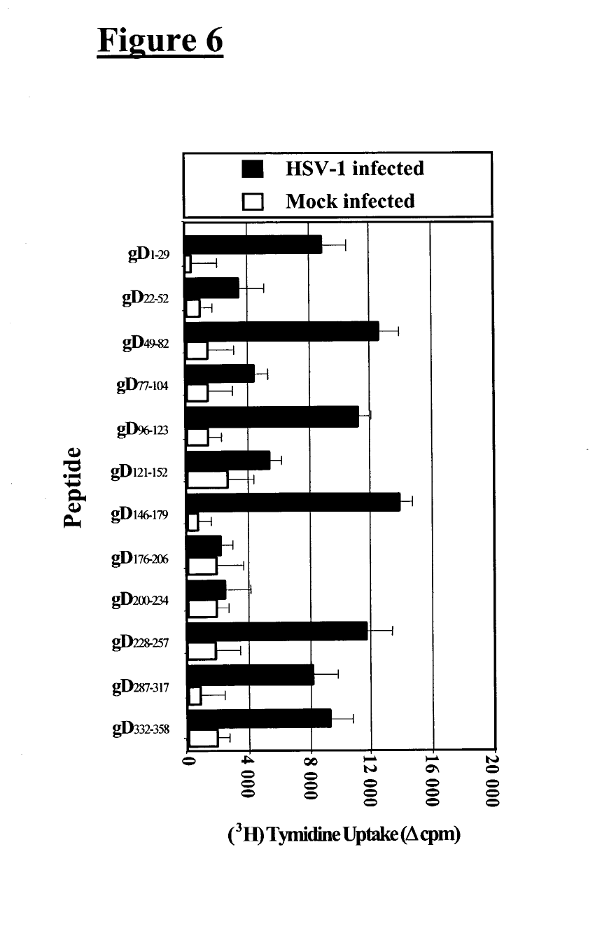

To define the fine specificity of broadly reactive T-cells associated with viral immunity and to explore immunodominance in the context of HSV infection, proliferation of lymphocytes obtained from twenty HSV-1 infected H-2^{d} mice were evaluated using the twelve gD peptides as Ag (FIG. 6). Although the selected peptides stimulated moderate HSV-specific T-cell responses, surprisingly, the HSV-primed T-cells were reactive to 8 to 10 of the 12 gD peptides, depending on the specific mouse, at the time of analysis. Despite a difference between individual mice, a unique array of T-cell responses was identified for each of the twenty infected mice analyzed. Seven peptides (gD₁₋₂₉, gD₄₉₋₈₂, gD₉₆₋₁₂₃, gD₁₄₆₋₁₇₉, gD₂₂₈₋₂₅₇, gD₂₈₇₋₃₁₇ and response in more then 85% of the HSV-infected mice (FIG. 6). The responses were found to gD₁₋₂₉, gD₄₉₋₈₂, gD₁₄₆₋₁₇₉, gD₂₈₇₋₃₁₇ and gD₃₃₂₋₃₅₈ immunodominant epitopes, also to gD₂₂₋₅₂, gD₇₇₋₁₀₄, gD₉₆₋₁₂₃ and gD₁₂₁₋₁₅₂, that represent subdominant epitopes in H-2^{d} mice. No correlation was found between the affinity of the peptides to MHC class II molecules and their ability to induce a T-cell response. Indeed, consistent with their ability to bind I-E^{d} molecule, gD₁₋₂₉ and gD₁₄₆₋₁₇₉ recalled high T-cell responses in HSV infected H-2^{d} mice (FIG. 6). However, gD₇₇₋₁₀₄, gD₂₀₀₋₂₃₄ and gD₂₈₇₋₃₁₇, that are also strong binders of I-E^{d} molecules, induced either low or no response (FIG. 6). Together these results indicate that the predicted regions contain epitopes that are naturally processed and presented to host's immune system during the course of HSV infection.

### Example 17

Determination of Ability of a Pool of Identified gD-peptide Epitopes to Survive a Lethal HSV-1 Challenge

The gD₄₉₋₈₂, gD_{146-179,} gD₂₂₈₋₂₅₇ and gD₃₃₂₋₃₅₈ peptides were tested for their ability to provide protective immunity against a lethal challenge with HSV-1 as depicted in Table 3. In these experiments, the pools were favored to individual peptides as they elicited higher levels of T-cell responses (FIG. 3). These four peptide epitopes (excluding the previously described protective epitope gD₁₋₂₉) were selected as they were found: i) to generate potent CD4⁺ T-cell responses in mice of diverse MHC background, ii) to elicit the strongest IL-2 and IFN-γ production, and iii) to induce T-cells that recognized native viral protein as presented by HSV-1-infected bone marrow derived-dendritic cells, and iv) to recall T-cell response in HSV-1 infected mice.

TABLE III
Immunization with newly identified gD peptide epitopes in the
Montanide's ISA-720 adjuvant confers protective immunity from a lethal
HSV-1 challenge^{(a)}

| | % of | | | | p versus^{(c)} |
|---|---|---|---|---|---|
| Mice | Spleen cells | | No. Protected/ | % of^{(b)} | gD vaccinated |
| injected with | CD4⁺ | CD8⁺ | No. Tested | Protection | mice |
| gD peptides | 18.1 | 5.6 | 10/10 | 100% | |
| Montanide | 16.3 | 5.1 | 1/10 | 10% | p = 0.0001 |
| None | 15.3 | 4.6 | 1/10 | 10% | p = 0.0001 |

^{(a)}Age and sex matched H-2^{d} mice were immunized with gD₄₉₋₈₂, gD₁₄₆₋₁₇₉, D_{228-257 and gD}₃₃₂₋₃₅₈ peptides emulsified in Montanide's ISA 720 adjuvant, injected with Montanide's ISA 720 alone, or left untreated (None). Mice were subsequently challenged with HSV-1 (10⁵ pfu/eye) and monitored daily for lethality
^{(b)}Results are representative of two independent experiments.
^{(c)}p values comparing the vaccinated mice to the adjuvant injected or non-immunized mice using Student's t test.

Groups of ten H-2^{d} mice were immunized with a pool of gD₄₉₋₈₂, gD₁₄₆₋₁₇₉, gD₂₂₈₋₂₅₇ and gD₃₃₂₋₃₅₈ emulsified in M-ISA-720 adjuvant, injected with M-ISA-720 alone (adjuvant injected control), or left untreated (non-immunized control). Mice were followed for four weeks for their ability to withstand a lethal infection with the McKrae strain of HSV-1. All of the mice that died following challenge did so between day 8 and 12 post-infection. All of the H-2^{d} mice immunized with the pool of gD peptides survived the lethal HSV-1 challenge. In contrast, only 10% of adjuvant-injected and 10% of non-immunized control H-2^{d} mice survived the HSV-1 challenge (Table 3). In a subsequent experiment, H-2^{d} mice immunized with a pool of the weak immunogenic peptides (gD₂₂₋₅₂, gD₇₇₋₁₀₄, gD₁₂₁₋₁₅₂ and gD₂₀₀₋₂₃₄) were comparatively more susceptible to lethal ocular HSV-1 infection (i.e. less then 50% survival).

To determine the involvement of CD4⁺ and CD8⁺ T-cells in the induced protection, mice were immunized with gD₄₉₋₈₂, gD₁₄₆₋₁₇₉, gD₂₂₈₋₂₅₇ and gD₃₃₂₋₃₅₈ peptides and then divided into four groups of ten. The groups were then depleted of CD4⁺ T-cells, depleted CD8⁺ T-cells, left untreated (none), or treated with irrelevant antibodies (rat IgG; IgG control). All four groups were then challenged with HSV-1 as described above. Depletion of CD4⁺ T-cells resulted in the death of all infected mice, indicating a significant abrogation of protective immunity as depicted in Table 4. However, depletion of CD8⁺ T-cells or injection of control rat IgG antibodies did not significantly impair the induced protective immunity (p=0.47 and p=1, respectively) (Table 4). These results demonstrate that, in this system, CD4⁺ T-cells are required and CD8⁺ T-cells are not required for protective immunity against lethal HSV-1 challenge.

TABLE IV
Immunization with the newly identified gD peptide epitopes in
the Montanide adjuvant induced a CD4+ T-cell-dependent protective
immunity against a lethal HSV-1 challenge^{(a)}

| Imunnized | % of | | No. | | p versus^{(c)} |
|---|---|---|---|---|---|
| mice | Spleen cells | | Protected/ | % of^{(b)} | gD vaccinated |
| treated with | CD4⁺ | CD8⁺ | No. Tested | Protection | untreated mice |
| None | 14.3 | 5.3 | 10/10 | 100% | |
| Anti-CD4 mAb | 0.3 | 4.1 | 0/10 | 0% | p = 0.0001 |
| Anti-CD8 mAb | 18.1 | 0.06 | 8/10 | 80% | p = 0.47 |
| IgG control | 14.7 | 6.7 | 9/10 | 90% | p˜1 |

^{(a)}gD vaccinated H2^{d} mice were left untreated (None) or depleted of CD4⁺ or CD8⁺ T cells by i p. injections of corresponding mAbs. Control mice received i.p. injections with a rat IgG.
^{(b)}Results are representative of two independent experiments.
^{(c)}p values comparing the vaccinated untreated mice to the anti-CD4 mAb, anti-CD8 mAbs or IgG treated mice as determined using student's t test.

While the description above refers to particular embodiments of the present invention, it will be understood that many modifications may be made without departing from the spirit thereof. For instance, the peptides of the present invention may be used in the treatment of any number of variations of HSV where observed, as would be readily recognized by one skilled in the art and without undue experimentation. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A vaccine composition, comprising:
a Herpes Simplex Virus (HSV) glycoprotein D (gD) peptide epitope; and
a pharmaceutical carrier.

2. The composition of claim 1, wherein the HSV gD peptide epitope is identified using an epitope algorithm analysis of gD.

3. The composition of claim 2, wherein the epitope algorithm is a TEPITOPE algorithm.

4. The composition of claim 1, wherein the HSV gD peptide epitope is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, peptide epitopes including SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, portions thereof and combinations thereof.

5. The composition of claim 1, wherein the pharmaceutical carrier is selected from the group consisting of water, an alcohol, a natural or hardened oil, a natural or hardened wax, a calcium carbonate, a sodium carbonate, a calcium phosphate, kaolin, talc, lactose and combinations thereof.

6. The composition of claim 1, further comprising an adujvant.

7. The composition of claim 1, further comprising from about 50 μg to about 5 mg of the HSV gD peptide epitope.

8. The composition of claim 1, further comprising from about 10 μL to about 100 μL of the pharmaceutical carrier.

9. The composition of claim 6, further comprising from about 15 μL to about 25 μL Montanide ISA720.

10. The composition of claim 1, wherein the composition is formulated to be administered by a technique selected from the group consisting of systemic injection, mucosal administration, topical administration, spray, drop, aerosol, gel, sweet formulation and combinations thereof.

11. The composition of claim 1, wherein the composition is formulated for delivery performed at an interval of about every four to six weeks.

12. The composition of claim 1, further comprising an additional component selected from the group consisting of a vehicle, an additive, an excipient, a pharmaceutical adjunct, a therapeutic compound or agent useful in the treatment of HSV and combinations thereof.

13. The composition of claim 1, wherein the composition is effective in the treatment of a condition selected from the group consisting of HSV, HSV-1 primary infections, HSV-1 recurrences, HSV-2 primary infections, HSV-2 recurrences, cold sores, genital lesions, corneal blindness, and encephalitis, a condition in which an expansion of CD4⁺ T-cells is desirable, a condition in which a stimulation of IL-2 is desirable, a condition in which a stimulation IFN-γ is desirable, a condition in which the induction of the Th-1 subset of T-cells is desirable and combinations thereof.

14. A method of treating a Herpes Simplex Virus (HSV) epitope-sensitive condition, comprising:
administering to a mammal an effective amount of a composition, comprising:
an HSV glycoprotein D (gD) peptide epitope; and
a pharmaceutical carrier.

15. The method of claim 14, wherein the HSV epitope-sensitive condition is selected from the group consisting of HSV, HSV-1 primary infections, HSV-1 recurrences, HSV-2 primary infections, HSV-2 recurrences, cold sores, genital lesions, corneal blindness, and encephalitis, a condition in which an expansion of CD4⁺ T-cells is desirable, a condition in which a stimulation of IL-2 is desirable, a condition in which a stimulation IFN-γ is desirable, a condition in which the induction of the Th-1 subset of T-cells is desirable and combinations thereof.

16. The method of claim 14, wherein the HSV gD peptide epitope is identified using an epitope algorithm analysis of gD.

17. The method of claim 16, wherein the epitope algorithm is a TEPITOPE algorithm.

18. The method of claim 14, wherein the HSV gD peptide epitope is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, peptide epitopes including SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, portions thereof and combinations thereof.

19. The method of claim 14, wherein the pharmaceutical carrier is selected from the group consisting of water, an alcohol, a natural or hardened oil, a natural or hardened wax, a calcium carbonate, a sodium carbonate, a calcium phosphate, kaolin, talc, lactose and combinations thereof.

20. The method of claim 14, wherein the composition further comprises an adjuvant.

21. The method of claim 14, wherein the composition further comprises from about 50 μg to about 5 mg of the HSV gD peptide epitope.

22. The method of claim 14, wherein the composition further comprises from about 10 μL to about 100 μL of the pharmaceutical carrier.

23. The method of claim 20, wherein the composition further comprises from about 15 μL to about 25 μL Montanide ISA720.

24. The method of claim 14, wherein administering to the mammal the effective amount of the composition further comprises using an administration technique selected from the group consisting of systemic injection, mucosal administration, topical administration, spray, drop, aerosol, gel, sweet formulation and combinations thereof.

25. The method of claim 14, wherein administering to the mammal the effective amount of the composition further comprises administering the composition about every four to six weeks.

26. The method of claim 14, wherein the composition further comprises an additional component selected from the group consisting of a vehicle, an additive, an excipient, a pharmaceutical adjunct, a therapeutic compound or agent useful in the treatment of HSV and combinations thereof.

27. A method for creating an epitope-based vaccine, comprising:
identifying an immunogenic epitope;
synthesizing a peptide epitope from the immunogenic epitope; and
creating a composition, comprising:
the peptide epitope, and
a pharmaceutical carrier.

28. The method of claim 27, wherein identifying an immunogenic epitope further comprises implementing an algorithm.

29. The method of claim 28, wherein the algorithm is a TEPITOPE algorithm.

30. The method of claim 27, wherein the immunogenic epitope is at least a portion of a Herpes Simplex Virus (HSV) glycoprotein.

31. The method of claim 27, wherein the immunogenic epitope is at least a portion of an HSV glycoprotein D (gD).

32. The method of claim 27, wherein the peptide epitope is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, peptide epitopes including SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, portions thereof and combinations thereof.

33. The method of claim 27, wherein the pharmaceutical carrier is selected from the group consisting of water, an alcohol, a natural or hardened oil, a natural or hardened wax, a calcium carbonate, a sodium carbonate, a calcium phosphate, kaolin, talc, lactose and combinations thereof.

34. The method of claim 27, wherein the composition further comprises an adjuvant.

35. The method of claim 27, wherein the composition further comprises from about 50 μg to about 5 mg of the peptide epitope.

36. The method of claim 27, wherein the composition further comprises from about 10 μL to about 100 μL of the pharmaceutical carrier.

37. The method of claim 27, wherein the composition further comprises from about 15 μL to about 25 μL Montanide ISA720.

38. The method of claim 27, wherein the composition is formulated to be administered with a technique selected from the group consisting of systemic injection, mucosal administration, topical administration, spray, drop, aerosol, gel, sweet formulation and combinations thereof.

39. The method of claim 27, wherein the composition is formulated for delivery performed at an interval of about every four to six weeks.

40. The method of claim 27, wherein the composition further comprises an additional component selected from the group consisting of a vehicle, an additive, an excipient, a pharmaceutical adjunct, a therapeutic compound or agent useful in the treatment of HSV and combinations thereof.
